(19) United States
(12) Patent Application Publication
(10) Pub. No.: US 2025/0176968 A1
(43) Pub. Date: Jun. 5, 2025

(54) TETHERED COMPLIANCE-ENHANCEMENT BALLOONS

(71) Applicant: EDWARDS LIFESCIENCES CORPORATION, Irvine, CA (US)

(72) Inventor: Yaron Herman, Givat Ada (IL)

(73) Assignee: EDWARDS LIFESCIENCES CORPORATION, Irvine, CA (US)

(21) Appl. No.: 19/047,425

(22) Filed: Feb. 6, 2025

Related U.S. Application Data
(63) Continuation of application No. PCT/US2023/071240, filed on Jul. 28, 2023.
(60) Provisional application No. 63/371,282, filed on Aug. 12, 2022.

(51) Int. Cl. A61B 17/12

(52) U.S. Cl. CPC A61B 17/12136; A61B 17/12036; A61B 17/12109

(57) ABSTRACT

A compliance-enhancing medical implant device includes a frame having an axially bulging cylindrical form in an expanded configuration of the frame and a radially-expandable tube disposed within the frame. Compliance-enhancing medical implant devices are further disclosed that comprise a balloon chain including a plurality of compressible balloons connected in series, a tether coupled to a proximal-most one of the plurality of compressible balloons, and an anchor means coupled to a proximal end of the tether.

### RELATED APPLICATION(S)

This application is a continuation of International Patent Application No. PCT/US23/71240, filed Jul. 28, 2023, which claims the benefit of U.S. Provisional Patent Application Ser. No. 63/371,282, filed on Aug. 12, 2022, the complete disclosures of which are hereby incorporated by reference in their entireties.

### BACKGROUND

### Field

The present disclosure generally relates to the field of medical implant devices.

### Description of Related Art

Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and systems that facilitate the restoration of compliance characteristics to undesirably stiff blood vessels. Devices associated with the various examples of the present disclosure can include one or more compressible balloons tethered to an anchoring structure, wherein the balloon(s) is/are configured to change in volume as environmental pressure conditions change, thereby evening-out blood pressure in a target blood vessel (e.g., aorta) in which the balloon(s) is/are implanted over the course of the cardiac cycle. The compliance-enhancing balloon(s) may be tethered to an anchor disposed in the target blood vessel or in a ventricle of the patient's heart, in which case the tether may pass through a valve of the heart (e.g., aortic valve).

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates example cardiac and vascular anatomy of a patient.

FIG. 2A shows an example healthy aorta.

FIGS. 2B and 2C show side and axial cross-sectional views, respectively, of the healthy aorta of FIG. 2A experiencing compliant expansion and contraction over a cardiac cycle.

FIG. 3A shows an example stiff aorta.

FIGS. 3B and 3C show side and axial cross-sectional views, respectively, of the stiff aorta of FIG. 3A experiencing compromised expansion and contraction over a cardiac cycle.

FIGS. 4A and 4B show side and axial views, respectively, of a compliance-enhancing balloon implant device in an expanded configuration in accordance with one or more examples.

FIGS. 5A and 5B show side and axial views, respectively, of a compliance-enhancing balloon implant device in a compressed configuration in accordance with one or more examples.

FIG. 6 shows a vacuum-sealed compliance-enhancing balloon including a structural frame in accordance with one or more examples.

FIG. 7 shows a balloon-chain compliance-enhancing implant device deployed in an aorta and anchored in a left ventricle in accordance with one or more examples.

FIG. 8 shows a balloon-chain compliance-enhancing implant device in accordance with one or more examples.

FIG. 9 shows a tethered compliance-enhancing balloon device in accordance with one or more examples.

FIG. 10 shows a balloon-chain compliance-enhancing implant device deployed in various positions in accordance with one or more examples.

FIG. 11 shows a balloon-chain compliance-enhancing implant device anchored/tethered to a prosthetic valve frame in accordance with one or more examples.

FIGS. 12-1, 12-2, and 12-3 illustrate a flow diagram for a process for implanting a compliance-enhancing implant device in accordance with one or more examples.

FIGS. 13-1, 13-2, and 13-3 provide images of the compliance-enhancing implant device and certain anatomy corresponding to operations of the process of FIGS. 12-1, 12-2, and 12-3 according to one or more examples.

FIG. 14 shows a balloon-chain compliance-enhancing implant device deployed in a pulmonary artery and anchored in a right ventricle in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, compliance-enhancement implant devices implanted in the aorta. However, although certain principles disclosed herein may be particularly applicable to the anatomy of the aorta, it should be understood that compliance-enhancement implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

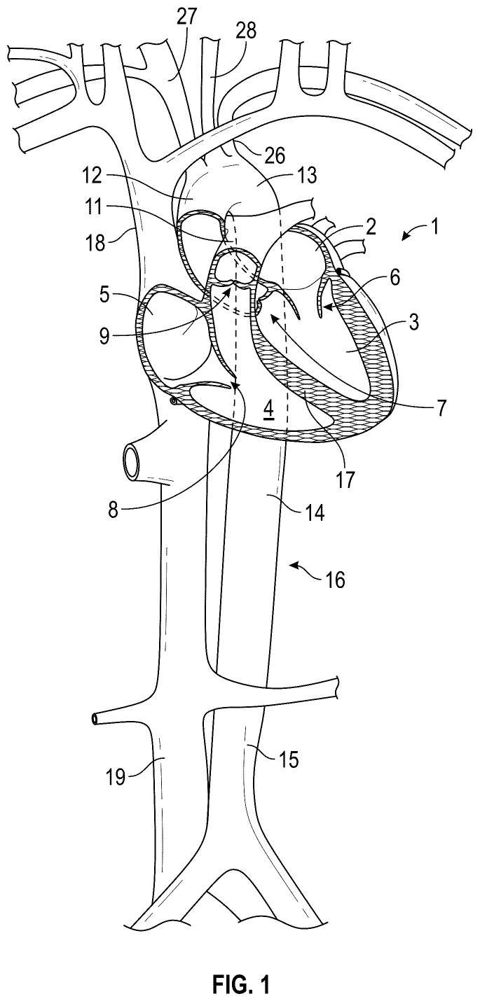

FIG. 1 illustrates an example representation of a heart 1 and associated vasculature having various features relevant to one or more examples of the present inventive disclosure. The heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16 is a compliant arterial blood vessel that buffers and conducts pulsatile left ventricular output and contributes the largest component of total compliance of the arterial tree. The aorta 16 includes the ascending aorta 12, which begins at the opening of the aortic valve 7 in the left ventricle of the heart. The ascending aorta 12 and pulmonary trunk 11 twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from ascending aorta 12 to aortic arch 13 is at the pericardial reflection on the aorta. At the root of the ascending aorta 12, the lumen has three small pockets between the cusps of the aortic valve and the wall of the aorta, which are called the aortic sinuses or the sinuses of Valsalva. The left aortic sinus contains the origin of the left coronary artery and the right aortic sinus likewise gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

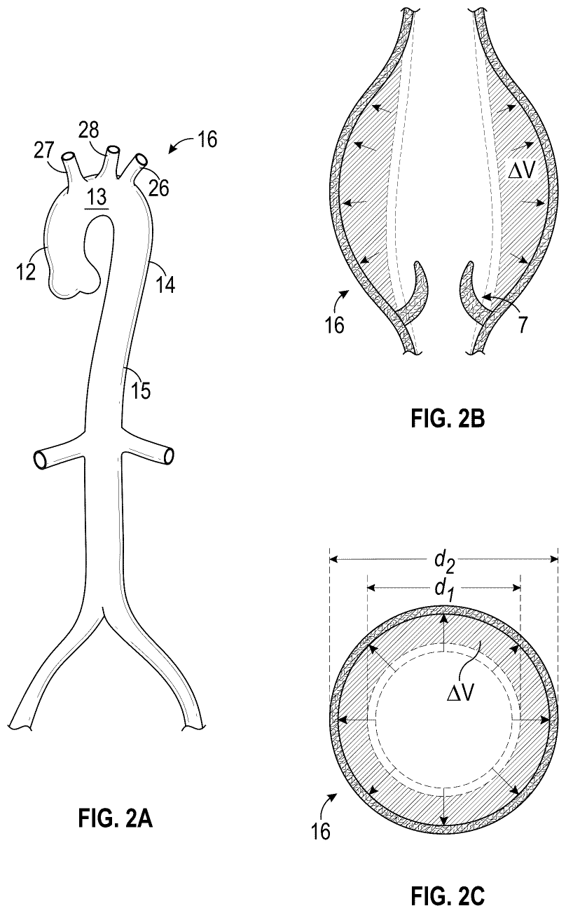

FIG. 2A shows an example healthy aorta 16. FIGS. 2B and 2C show side and axial cross-sectional views, respectively, of the healthy aorta 16 of FIG. 2A experiencing compliant expansion and contraction over a cardiac cycle.

As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

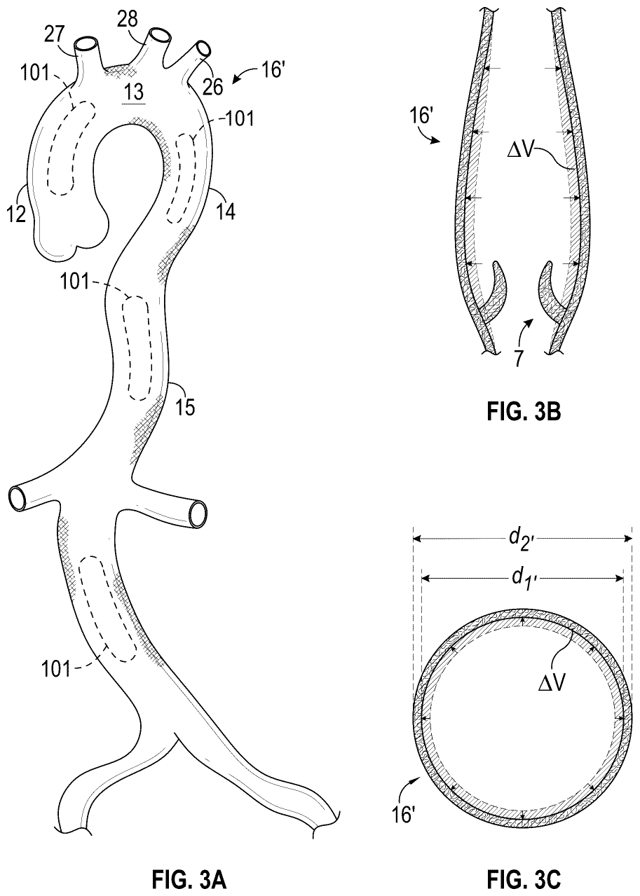

FIG. 3A shows an example stiff aorta 16′. FIGS. 3B and 3C show side and axial cross-sectional views, respectively, of the stiff aorta 16′ of FIG. 3A experiencing compromised expansion and contraction over a cardiac cycle.

As shown in FIG. 3A, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter d₁′ to the expanded diameter d₂′ may be limited/reduced compared to the expansion of diameter of a healthy blood vessel. Although FIGS. 3B and 3C show a small amount of expansion and volume change Δv′ experienced by the blood vessel 16′, in some cases, a blood vessel may be sufficiently stiff that substantially no vessel expansion takes place in systole.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing, tethered balloon implant devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system. Examples of the present disclosure provide compliance-enhancing tethered balloon implant devices, which may be implanted in one or more locations in a compromised aorta and/or other vessel(s). For example, FIG. 3A shows example positions of tethered balloon devices 101 including features disclosed herein implanted in various areas of an aorta 16′.

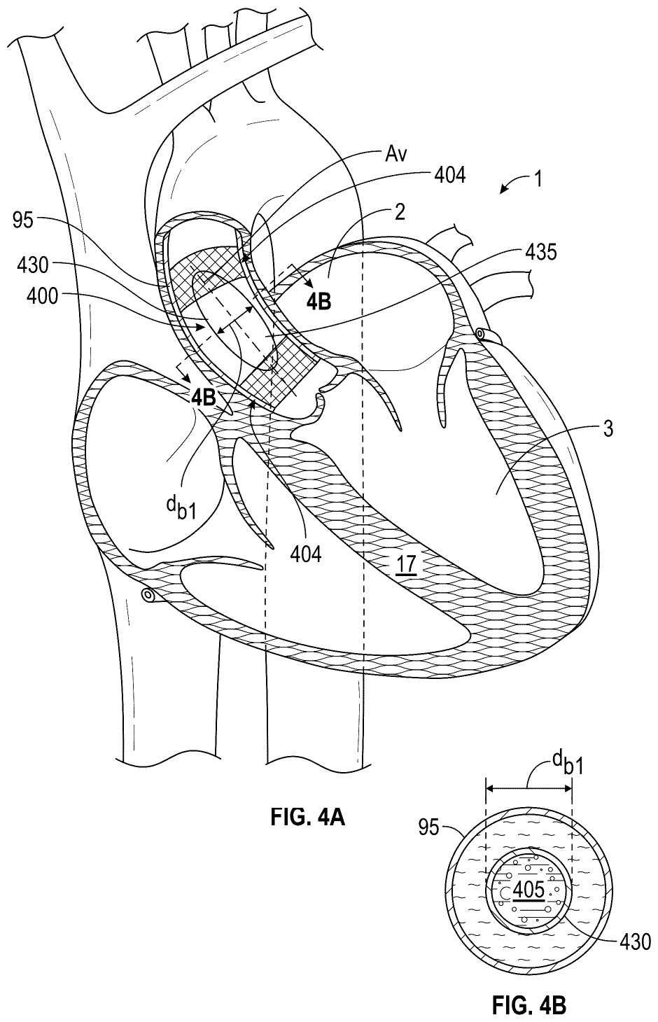

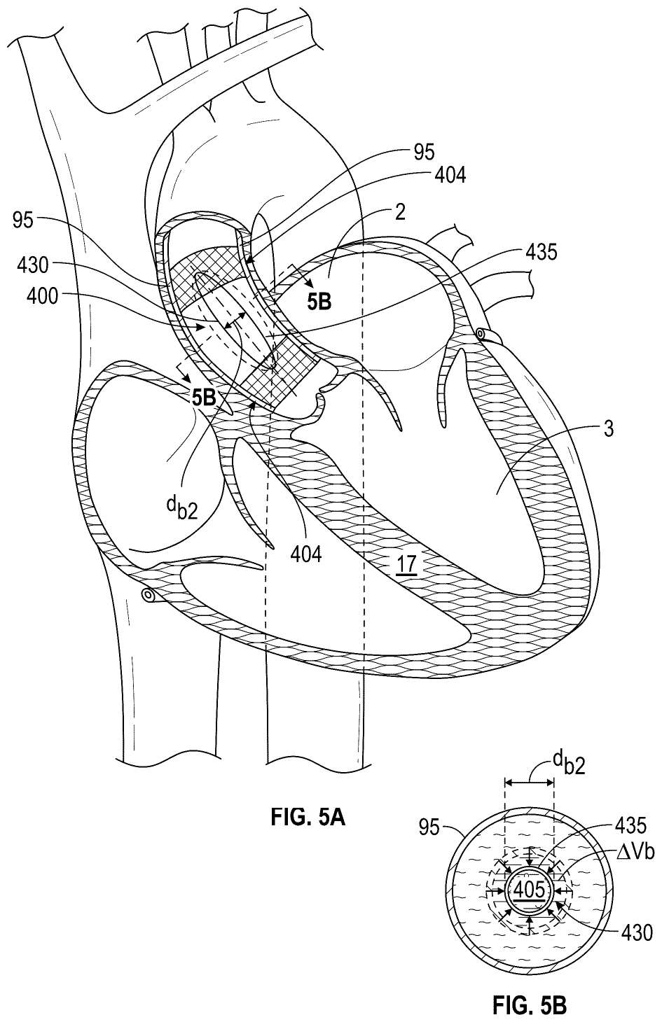

FIGS. 4A and 4B show side and axial views, respectively, of a compliance-enhancing balloon implant device 400 in an expanded configuration in accordance with one or more examples. FIGS. 5A and 5B show side and axial views, respectively, of the compliance-enhancing balloon implant device 400 of FIGS. 4A and 4B in a compressed configuration in accordance with one or more examples.

The implant device 400 comprises a volume/form configured to become compressed and/or reshaped in response to increasing fluid pressure in a blood vessel or other chamber 95 in which the implant 400 is implanted. For example, the implant 400 may include a spheroid, cylindrical, or other-shaped form/volume configured such that pressure against/on an outer surface thereof causes the device to compress and/or reshape from a form having a first expanded diameter d_{b1} to a compressed diameter d_{b2} (shown in FIG. 4B), wherein subsiding pressure conditions allow for and/or cause the device form 430 to re-expand to the expanded diameter d_{b1}. Such alternation between the expanded d_{b1} and compressed d_{b2} diameters can result in a change in volume Δv_{b} occupied by the balloon component 430 of the device 400 in correlation with changing pressure conditions, wherein such change in volume can reduce systemic pressure and/or flow during high-pressure conditions (e.g., systole) and increase fluid pressure and/or flow during lower-pressure conditions (e.g., diastole). Such effects on the systemic flow in the vessel 95 can increase compliant characteristics of the blood vessel 95 in the case where the compliance thereof has been compromised due to aging and/or other conditions. When compressed, as shown in FIGS. 5A and 5B, the device 400 may store energy associated with the biasing of the shape thereof toward the expanded configuration shown, such that such energy may be returned to the blood circulation as pressure subsides, thereby increasing flow therein.

The implant device 400 is shown as an example axial compliance-enhancing implant device, wherein the balloon 430 of the device 400 is aligned with and/or overlaps a central axis Aᵥ of the blood vessel 95, such that blood flow through the portion of the blood vessel 95 shown passes through the blood vessel in the area around the spheroid form 430. Although shown and described as an axial implant device, it should be understood that compressible devices similar to the device 400 may be implanted within a target blood vessel in a non-overlapping position with respect to the axis Aᵥ of the blood vessel 95. For example, the implant 400 may be positioned against a wall of the blood vessel 95, wherein blood flow through the vessel passes over and/or around the outside of the balloon 430 of the implant device 400. Any of the example compliance-enhancing implant devices of the present disclosure may be similar in one or more respects to the implant device 400, with respect to shape, configuration, components, and/or other aspects/features thereof.

The implant device form/structure 430 may be biased in the expanded state shown in FIGS. 4A and 4B by gas or other media/medium contained therein, wherein such media can be compressed/compressible under relatively high pressures. Additionally or alternatively, the form of the device 400 may be biased in the expanded configuration by mechanical attributes of the form/frame 430, which may hold the device 400 in the expanded configuration in the absence of sufficient external fluid pressure. For example, a wireframe or other structure may form the spheroid shape of the device 400, wherein the frame may be covered by a fluid-tight covering of the device 400, the device 400 may be vacuum-filled, or may comprise certain compressible media. The cover 435 of the device 400 may or may not be elastic.

The implant 400 may be anchored within the target blood vessel 95 in any suitable or desirable manner. For example, in some implementations, as shown in FIGS. 4A and 5A, one or more stents or other anchors 404 may be deployed within the blood vessel 95, wherein such anchors hold the implant 400 in the desired position within the blood vessel 95, such as within a central area/region within the vessel. The anchor(s) 404 may advantageously be designed to the deployed within the blood vessel 95 without substantially impeding the flow of blood around the balloon form 430.

Generally, the greater the diameter d_{b1} of the balloon 430, the greater the available/possible volume change and compliance provided by the balloon 430. However, compliance balloons as shown in FIG. 4A can obstruct flow within the blood vessel 95 to some degree. Therefore, implementing balloon implants having relatively greater diameters tends to obstruct blood flow to a greater extent, which can cause stenosis, constriction, and/or other detrimental effects. In some implementations, example compliance balloon devices of the present disclosure divide the total balloon volume among a plurality of separate, serially-connected balloons, which may tend to obstruct blood flow through the vessel to a lesser extent than implementations in which the total volume of an balloon implant is embodied in a single spheroid/ellipsoid balloon.

The balloon 430, as with any of the compliance-enhancing balloon devices disclosed herein, can comprise compressible gas or other compressible medium/media in the internal space 405 thereof, wherein compression of such media provides the change in volume and stored energy for introducing compliance to the blood vessel in which the device is implanted. In such implementations, the compression threshold pressure and/or the rate/degree of compression of the balloon in certain pressure conditions may be controlled at least in part by the type and/or volume of gas or other medium/media disposed therein. Compressible gases used in compliance-enhancing balloon devices of the present disclosure can be any inert and/or biocompatible gas. In some implementations, carbon dioxide gas is used.

In some implementations, compliance-enhancing balloon devices of the present disclosure can be vacuum-sealed, wherein the space within the balloon comprises a vacuum. Such compliance-enhancing balloon devices may include an inner or outer frame configured to hold the balloon shape, wherein a covering/sealing seals the space within the frame in a manner that holds a vacuum space/volume therein.

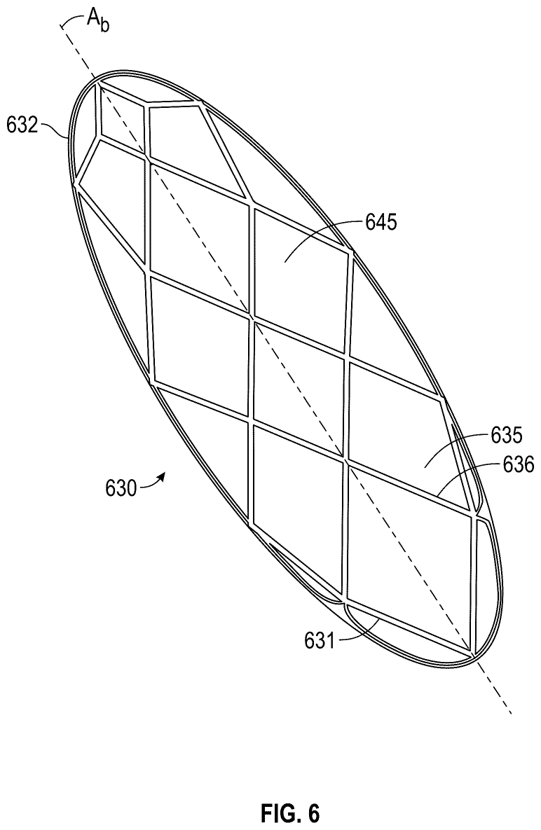

FIG. 6 shows a vacuum-sealed compliance-enhancing balloon 630 including a structural frame 631 in accordance with one or more examples. The frame 631 may serve as a chamber-support structure for maintaining a spheroid or other shape having a vacuum therein. The frame 631 may comprise braded wire mesh or other wire support form. The frame 631 may have a shape-memory biased shape in the spheroid or other form, but may be compressible for placement within a delivery catheter/sheath, wherein deployment from the catheter/sheath enables shape-memory expansion of the frame 631.

The balloon 630 includes a fluid-tight covering 632, which prevents ingress and/or egress of fluid into and/or out of the internal chamber 645 of the balloon 630. The balloon frame 631 may serve as a mechanical support for the balloon 630, wherein the frame 631 is at least partially covered (internally and/or externally) by the covering 632, which may comprise polymer or cloth. The frame 631 may be configured to exert outward force on an inner surface of the balloon covering 632 to prevent or reduce collapsing of the vacuum chamber 645. The inner balloon chamber 645 may be sealed to form an internal volume that provides the compliance chamber for the balloon 630.

The balloon frame 631 may comprise self-expanding memory metal (e.g., Nitinol). The balloon 630 may advantageously be designed to have a length and/or diameter sufficient to provide the desired volume, while maintaining a diameter small enough to avoid detrimental obstruction of blood flow. The frame 631 can comprise a plurality of wires/struts configured to hold the expanded shape of the balloon 630 and to deflect inwardly and/or towards alignment with the axis A_{b} of the balloon 630 in response to ambient pressure forces. Although metal strut-based frames are described in connection with certain examples of the present disclosure, it should be understood that any type of support structure may be implemented, wherein the structure, such as struts, or the like, are configured to store and release energy in response to changes in ambient pressure.

The balloon 630 may be coupled to a tether and/or anchor device/structure to facilitate implantation thereof within a blood vessel lumen. For example, the balloon 630 may be coupled to one or more stent anchors configured for deployment within a blood vessel. In some examples, the balloon 630 has a tether wire, suture, or other line, coupled thereto, wherein the tether is configured to be anchored to blood vessel or other cardiac tissue. For example, the tether may have a tissue anchor at an end thereof (e.g., corkscrew anchor), or the tether may be sutured to tissue.

The frame 631 may provide a spring-like compliant frame having a geometrical shape designed to collapse into the vacuum volume 645 of the balloon 630 at certain pressure levels. Generally, systolic blood pressure in most patients is between 90-120 mmHg, whereas diastolic pressure is generally between 60-80 mmHg. Therefore, it may be desirable to construct the frame 631 (or, with respect to compliance-enhancing balloons disclosed herein that comprise compressible gas/medium without a structural support frame, select the type and/or volume of the compressible gas/medium) in a manner such that the frame 631 and/or covering 632 deflects/compresses radially inwardly in the presence blood pressure that exceeds a threshold level between 60-100 mmHg, such as between 80-90 mmHg (e.g., approximately 85 mmHg). That is, the various parameters of the frame 631 may be designed to provide compression and recoil on respective sides of a threshold pressure level that is between the lowest diastolic pressure and the highest systolic pressure level. The threshold pressure level may be selected as a neutral pressure, such that when the blood pressure is higher than the threshold, the balloon 630 compresses/contracts, whereas when the pressure is lower than the threshold and the frame is in the compressed/contracted state, the frame is inclined to expand/recoil back to the neutral expanded state.

The frame 631 may advantageously be designed with respect to various parameters thereof, which may include strut thickness, strut width, frame material, frame joint configuration, frame strut/cell design, shape-memory characteristics, number of struts, and/or other structural aspects of the frame geometry, such that relatively minimal pressure increase above the designed pressure threshold of the frame results in relatively substantial compression of the frame, which may advantageously reduce the amount of work required by the blood flow to cause compression of the frame. As an example, the frame 631 may have a diamond strut/cell pattern, or other strut/cell pattern, which produces a three-dimensional spring structure having a spring constant that is defined by the geometry of the frame 631. The ability to design the structure of the frame 631 in a manner as to produce the desired compression and expansion behavior provides desirable control over the performance of the balloon 630, which may be leveraged in a patient- and/or demographic-specific/customized manner.

The balloon 630 provides a sealing member 632 around the frame 631, which serves to prevent blood from entering the space/cavity within the balloon 630, while also holding an air-tight seal to allow for the presence of a vacuum condition within the balloon 630. Processes of the present disclosure may involve removing air/media from the internal cavity of the balloon 630 so as to produce the vacuum therein. Manufacturing of the balloon 630 may involve sealing the balloon 630 to maintain the vacuum within the balloon chamber 645. Sealing the balloon 630 may involve sealing the covering 632 around the frame 631 in a fluid-tight manner.

The covering/sealing 632 can provide a compliant outer layer. Although described as an outer layer in some contexts herein, it should be understood that the cover 632 may be disposed radially outside of the frame 631 and/or inside the frame 631. With the vacuum present within the balloons volume 645, the frame 631 alone and/or the frame in combination with the mechanical structure of the cover 632, may hold the three-dimensional shape of the balloon 630. By forcing a vacuum within the frame/balloon, the force/work required to collapse the frame 631, and therefore the balloon, can be reduced. That is, blood pressure outside of the balloon 630 may not be required to overcome compression forces of gas within the balloon 630, but rather only the mechanical resistance of the frame 631.

Although balloon devices are described herein that are configured to radially compress as a mechanism for changing in volume in response to changing pressure conditions, it should be understood that changing volume in balloon devices disclosed herein may be achieved using any suitable or desirable mechanism. For example, in some implementations, balloon devices of the present disclosure are configured to reshape in response to increasing and/or decreasing pressure external thereto in a manner as to change the volume of the balloon without necessarily requiring radial compression. Generally, for a balloon device having a generally circular cross-sectional shape in an expanded configuration thereof, deviation from the circular cross-sectional shape can result in a decreased cross-sectional area and/or volume of the balloon. Therefore, the frame 631 and/or other balloon structures or supports may be configured to transition from a more-circular cross-sectional shape to a less-circular cross-sectional shape in response to increasing pressure, thereby reducing the volume of the balloon as pressure increases, wherein the balloon may revert to the more-circular shape thereafter. Any compliance-enhancing balloon disclosed herein may be optionally a compressible-gas-filled balloon and/or a pressure-responsive reshaping balloon.

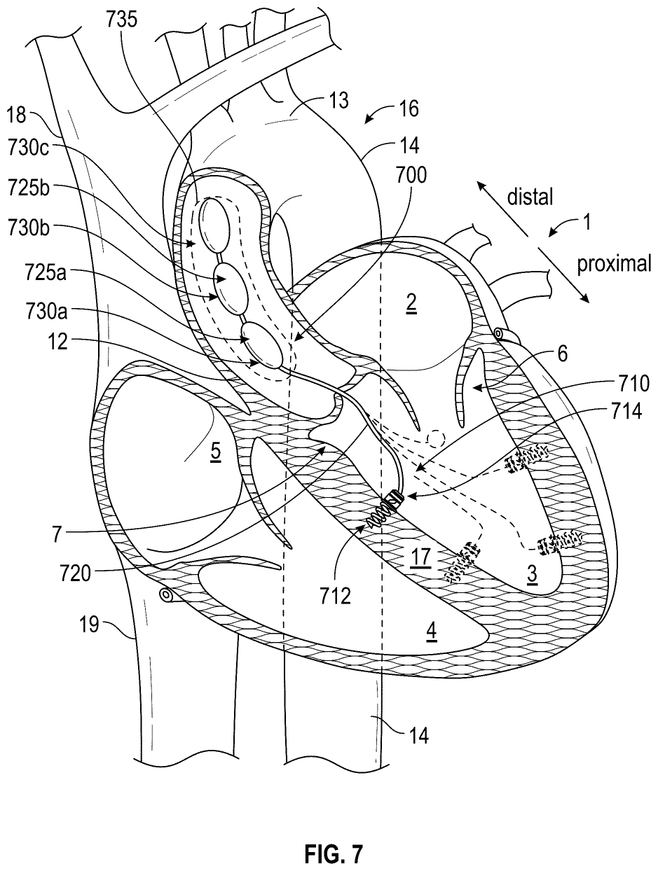

FIG. 7 shows a balloon-chain compliance-enhancing implant device 700 deployed in an aorta 16 and anchored in a left ventricle 3 in accordance with one or more examples. The implant device 700, as with other examples presented herein, can be implemented to add arterial compliance, which can advantageously reduce pulsatile left ventricular after flow, which may be helpful for hypertensive heart failure patients. The implant device 700, as with other examples of the present disclosure, may be implanted in patients suffering from aortic stiffness, wherein the aorta suffers from compromised compliance of arterial circulation. As compliance of the aorta accounts for a substantial or majority portion of the arterial circuit, reduced/compromised arterial compliance can cause increase in left ventricular afterload, which can lead to lower exercise tolerance, and other issues/complications. The implant device 700 may be implanted as a means to reduce arterial vessel compliance in, for example, congestive heart failure patients, in order to alleviate symptoms and/or reduce hospital readmissions. With the implant device 700 deployed at least partially within the arterial vessel, the implant device 700 may serve as a compliance-enhancing element to effectively reduce peak systolic pressure and/or arterial pulse pressure amplitude.

The implant device 700 comprises one or more compressible and/or re-shapable balloons 730, which are tethered to a tissue anchor 710. The anchor 710 may be anchored to any cardiac and/or vascular tissue. For example, in the illustrated implementation, the tissue anchor 710 is implanted/embedded in tissue associated with the left ventricle 3. For example, the tissue anchor 710 may be at least partially embedded and/or anchored to the ventricular septum 17. Although the anchor 710 is shown as anchored to the septum 17, it should be understood that the tissue anchor 710 may be anchored to any other tissue or anatomy of the left ventricle 3, as represented by the various dashed-line tissue anchor positions of FIG. 7. For example, the tissue anchor 710 may be embedded at least partially in and/or anchored to the outer ventricle wall, papillary muscle, chordate tendineae, trabeculae carneae, and/or valvular tissue (e.g., valve annulus), in anterior or posterior areas thereof.

In implementations in which the device 700 includes a plurality of compliance-enhancing balloon 730, such balloons may be connected in a series/chain arrangement, as shown. In some implementations, three or four balloons 730 may be connected in one or more groups 735 of balloons, referred to herein as a ‘balloon chain,’ or ‘balloon element.’ The balloons 730 may be tethered to the tissue anchor 710 by a tether line 720, which may comprise suture, wire, cloth/fabric, or any other line/band-type connector. The tissue anchor 710 may be coupled to a proximal end/portion of the tether line 720. The tether line 720 may be tethered/coupled to a proximal-most balloon 730a of the balloon element/chain 735. The tether line 720 may have any suitable length dimension. In some implementations, the tether is at least 3 cm in length. Such dimensions may allow for the tether 720 to span a distance from within the ventricle 3 to within the aorta 16 through the aortic valve 7.

Adjacent balloons of the balloon chain 735 may be coupled to one another by a connector 725, which may comprise any type of line, suture, wire, fabric/cloth, or the like. For example, as illustrated, the connector 725a connects the proximal-most balloon 730a to the second balloon 730b and the connector 725b connects the second connector 730b to the third, distal-most balloon 730c. In some implementations, the connectors 725 are integrated with frames of the connected balloons 730. The connector(s) 725 may comprise flexible or rigid struts (e.g., metal struts). The connectors 725 may be arranged/configured as axial and/or longitudinal connectors/lines between adjacent ones 730 of a plurality of the balloons 735.

Each of the balloons 730, as with any other examples of compliance-enhancing balloons disclosed herein, may comprise a polymer covering/material having characteristics that provide for fluid- and/or gas-barrier sealing. The balloons 730 may be inflated with a gas that is at least partially dissolvable in blood, such as CO₂. In some example, the balloons 730 are vacuum-filled. The balloons 730 may be configured to reshape and/or compress in response to pressure increases (e.g., pressures above 80 mmHg). Such compression and/or reshaping may involve reduction of a minor axis (with respect to the side view shown in FIG. 7) of the ovoid forms of the balloons 730.

The tissue anchor 710 may comprise any tissue-anchoring means. The anchor 710 may be formed at least in part of metal or other biocompatible material. In some examples, the anchor 710 is a corkscrew-type tissue anchor having a proximal drive head 714, which may be configured to be engaged by a driver tool to rotate and/or embed the anchor 710 into the target tissue. The anchor 710 may further include a tissue-engagement portion/feature 712, which may comprise a helical form or other barb, spike, or the like. In some examples, the tissue anchor 710 comprises a stainless-steel anchor that is between 5-10 mm in length (e.g., approximately 6 mm). The anchor 710 can be used to fasten the tether line/wire 720 to the target tissue (e.g., septum 17). Although a single tissue anchor is shown for the device 700, it should be understood that any number of tissue anchors may be implemented. The anchor 710 can be repositionable and/or retrievable prior to full deployment of the implant 700 as shown in FIG. 7. In some examples, the anchor 710 comprises suturing for suturing the tether line 720 to the cardiac tissue. In such examples, no other structural anchor may be used or required.

By implementing the compliance element 735 as a chain of balloons, the diameter of the balloons can be a relatively smaller for a given total volume of the balloon chain 735 compared to a single balloon having the same volume. Reducing the diameter/profile of the chain 735 can be advantageous to prevent undue obstruction of blood flow through the blood vessel 16. The total volume of the compliance-enhancing balloons/elements of the chain 735 may correspond to the sum of the individual volumes of the balloon 730, and therefore the total volume change experienced by the balloon chain 735 across low- and high-pressure stages/phases of the cardiac cycle may correspond to the sum of the volume changes of each of the balloons 730. As each of the balloons 730 has a relatively small profile relative to the total volume of the balloon chain 735, the profile of the compliance-enhancing elements 735 can be relatively small compared to the total volume of the combined balloons 730.

The tether line 720 may pass through the aortic valve 7 between the leaflets thereof. Therefore, it may be desirable for the tether 720 to be relatively thin so as to not unduly interfere with coaptation of the leaflets of the valve 7. In some implementations, a balloon or other structure may be coupled to the tether 720 in the area of the aortic valve 7 and may serve as a spacer that prevents aortic regurgitation. Due to the pressure/flow dynamics in the left side of the heart, the balloon chain 735 may advantageously be held within the aorta 16, rather than being inclined to migrate back through the aortic valve 7 into the left ventricle 3. That is, as the pressure in the aorta 16 is relatively high, and blood flow from the ventricle flows in the direction through the aortic valve 7 into the ascending aorta and through the aortic arch 13, the tether 720 and/or connector(s) 725 may generally be held in a taught tension, particularly in the systolic phase of the cardiac cycle when blood is pushed through the aortic valve 7. In some implementations, the device 700 may be initially deployed entirely within the left ventricle 3, wherein blood pressure and flow caused by squeezing of the left ventricle 3 may draw the compliance element/chain 735 through the aortic valve 7 and into the aorta 16 (e.g., the ascending aorta 12).

In certain examples, it may be desirable for the total volume change of the balloon element/chain 735 between diastole and systole to be between approximately 10-50 ml. Where the balloon(s) 735 is/are configured to collapse by a relatively greater volume, it may be possible to achieve desired compliance using a relatively smaller balloon(s). In some implementations, it may only be necessary for the balloon(s) 735 to collectively change in volume by 10-20 mL for patients with relatively better aortic compliance, whereas greater volume change may be desired for patients with stiffer aortas/vessels. In certain examples, the balloon(s) 735 may have a collective working volume of between 20-50 ml. Such specifications can advantageously achieve an increase in compliance from a value of approximately 0.9 to approximately 1.3 or greater.

Balloon compliance elements disclosed herein, such as the balloon chain 735, may have a length of approximately 15 cm, or less. In some implementations, compliance elements have a length of between 15-20 cm. In some implementations, balloon compliance elements have a length of 20 cm or greater. Compliance balloons of the present disclosure can be configured to have a compressed diameter of approximately 0.4 cm or less, or some other value. Compliance balloons of the present disclosure can be configured to have an expanded diameter of approximately 1.2 cm or less, or some other value. Compliance balloons of the present disclosure can be configured to have an expanded diameter of between approximately 1.2-2.0 cm. Compliance balloons of the present disclosure can be configured to have an expanded diameter of approximately 2.0 cm or greater. With longer balloons and/or balloon chains, it may be suitable to utilize an expanded diameter that is relatively less in order to provide the same effective working volume.

Compliance balloons of any of the examples disclosed herein may advantageously include relatively smooth outer surfaces/coatings to promote smooth fluid flow around such compliance elements and avoid thrombus build-up and/or interruptions to flow. Devices that unduly obstruct or resist flow of blood thereabout may cause static blood flow dynamics, wherein such static blood can promote thrombus/embolism formation, which can cause serious health risks.

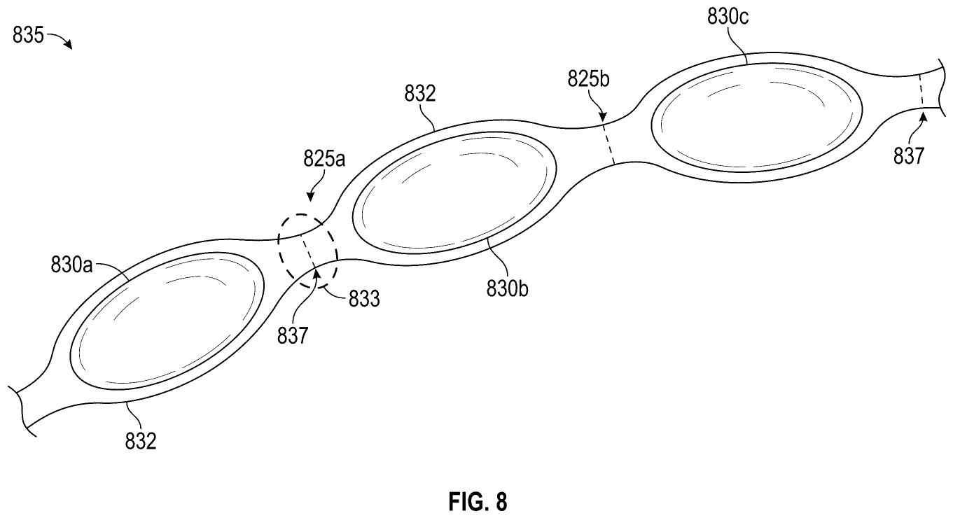

FIG. 8 shows a balloon-chain compliance-enhancing implant device 835 in accordance with one or more examples. The balloon chain 835 (also referred to in some contexts as a ‘compliance element’) may be tethered to a tissue anchor in accordance with any of the examples disclosed herein. The balloon chain 835 may have multiple compressible and/or re-shapable compliance-enhancing balloon segments or elements 830, which may be connected in a series/chain arrangement, as shown, by respective connector portions 825 of a cover 832 of the compliance element 835.

The balloon chain 835 includes an air-tight cover 832 that covers the compressible/re-shapable pockets/cavities 830 and connects between such elements to form the chain 835. The balloon pockets/cavities 830 may form a chamber or space, which may be filled with compressible gas or vacuum-filled. With respect to vacuum-filled implementations, the individual balloons 830 may comprise wireframes or the like configured to hold the ellipsoid or other shape of the balloon space without collapsing at low ambient pressures. In some implementations, the cover 832 may be fluid-sealed in areas 833 of the connector portions 825. For example, the walls of the cover 832 may be heat-sealed, crimped, or otherwise attached or bonded in such a manner as to prevent fluid of one balloon 830 from passing through the connector portion 825 of the cover 832 to another or to leak to the external environment.

In some implementations, a seam 837 in the sealed portion 833 may delineate adjacent balloons of the chain 835. Such seam 837 may have structural, visual, and/or other features associated therewith, or the seam may simply be understood to be any area within the sealed portion 833 where the chain can be cut or otherwise broken to selectively group/separate a desirable number of balloons 830 in a given chain 835. For example, the seam 837 may comprise one or more perforations that facilitate tearing, splitting, disconnecting, dividing, cutting, lacerating, cleaving, ripping, sectioning, etc., of the cover 832 along the seam 837 and/or in the area of the fluid-sealed cover portion(s) 833. The seam 837 may be orthogonal to a lengthwise dimension of the chain 835. Additionally or alternatively, the seam 837 may comprise one or more markings visually indicating to a user the area of the seam 837 where the chain 835 can be cut or otherwise broken in a manner as to group the desired number of balloons 830.

The cover 832 may comprise any suitable or desirable air-tight, biocompatible material, such as polymer, cloth, or the like. The ability to cut/break the chain 835 in any of the connector portions 825 of the cover 832 can allow for customization of the length and/or number of balloons of the chain 835, thereby allowing for patient-specific treatment. For example, where a greater amount of volume-changing compliance introduction is required, the chain 835 may be cut/broken in a manner as to include a greater number of balloon segments 830 (e.g., four or more balloons), whereas for some patients where a lesser degree of compliance enhancement is required, fewer balloon segments 830 may be selected (e.g., three, two, one). Furthermore, in some implementations where the volume of each of the balloons 830 is known, the total volume of the chain 835 can be calculated by summing the volumes of the selected number of balloons. For some patients, approximately 10-20 mL of volume, or volume change, over the cardiac cycle is suitable to provide the desirable compliance finality. Therefore, in implementations in which each balloon 830 comprises 5 mL of volume, or provides 5 mL of volume change between expanded and compressed configurations, 2-4 balloons may be selected, and the chain 835 may be broken/torn in a manner as to produce such length of the chain.

In some implementations, the balloons 830 do not include an internal frame, but rather may be circumferentially and axially sealed, such that a pocket/cavity formed by the cover 832 can be filled and maintained with compressible gas therein. Therefore, in some implementations, the balloon chain 835 may comprise only a single integrated structural element in the form of the cover 832, wherein the cover is configured with compressible-gas-filled pockets that are sealed and fluidly isolated from one another and from the external environment. In some implementations, the cover 832 is formed of more than one layer. For example, the coverage 32 may comprise two layers joined together in the sealed portions of the chain 835, thereby sandwiching the compressible gas chambers 830 between the two layers.

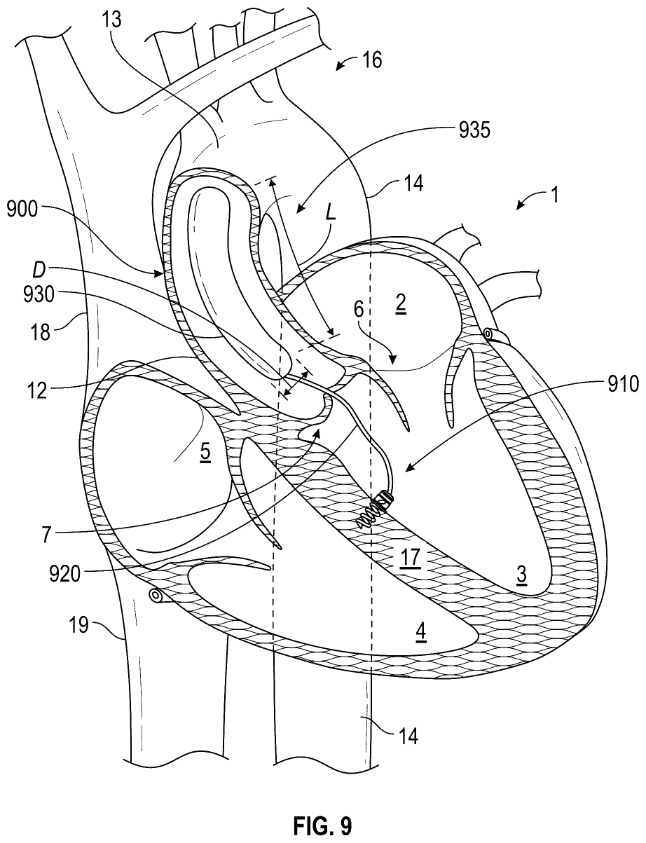

FIG. 9 shows a tethered compliance-enhancing balloon device 900 in accordance with one or more examples. The implant 900 includes a compliance element 935 tethered to an anchor 910 by a tethering line 920. Unlike the balloon-chain example shown in FIG. 7, the compliance element 935 of the device 900 may comprise a single elongated balloon as an alternative to a multi-balloon chain. As referenced above, it may be advantageous to reduce the profile of any compliance element implemented in connection with examples of the present disclosure.

While some examples disclosed herein achieve relatively low-profile structural form by dividing compliance elements into multiple serial/chained balloons, which achieves desirable balloon volume through the combination of multiple balloons, the implementation of FIG. 9 achieves relatively low profile and high volume by implementing an elongated ellipsoid form for the balloon 930 that has a length dimension L that is substantially greater than a diameter D the of the balloon 930. For example, the balloon may have a length L that is at least three times as great as the diameter D, or even four or five times greater, or more. The examples of the present disclosure may be implemented with a single elongated compliance-enhancing balloon as shown in FIG. 9, or two or more chained/connected elongated ellipsoid balloons. Compared to the single elongated balloon example of FIG. 9, multiple, chain-connected, smaller compliance balloons as shown in FIG. 7 and elsewhere, can provide greater control over the volume of the compliance element, such as by allowing for selection of the desired/suitable number of compliance balloons for a given balloon chain. Furthermore, balloon chain implementations may provide a relatively more conformal shape for the compliance element, allowing the compliance element to more easily/readily curve around bends in the vascular anatomy.

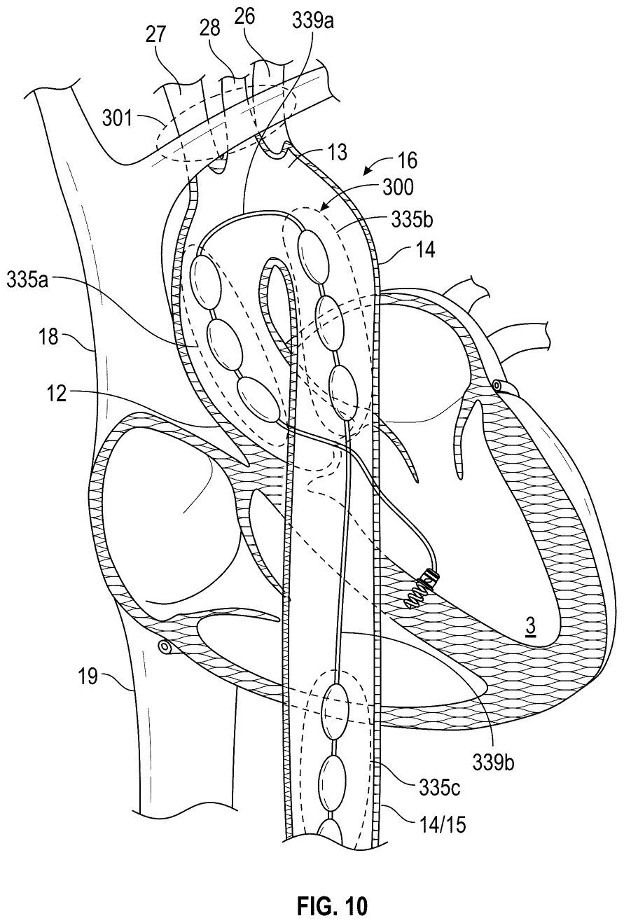

FIG. 10 shows a balloon-chain compliance-enhancing implant device 300 deployed in various positions in accordance with one or more examples. The illustration of FIG. 10 shows that compliance elements 335, which may represent any compliance element (e.g., balloon chain, elongated cylindrical or ellipsoid balloon) of any example of the present disclosure may be implanted in, or deployed in such a manner that the compliance element is disposed in, any suitable or desirable position or area within the target blood vessel (e.g., aorta 16). For example, as illustrated, compliance elements including one or more balloons (e.g., balloon chain) may be anchored/tethered such that they are deployed/disposed, after implantation, in the ascending aorta 12, aortic arch 13, descending aorta 14, descending thoracic aorta 14, descending abdominal aorta 15 (see FIG. 1; e.g., supra-renal or infra-renal portion of abdominal aorta), or other portion of a target blood vessel (e.g., inferior vena cava 19, pulmonary artery 11, superior vena cava 18).

As examples, FIG. 10 shows a first compliance element 335a deployed/disposed in the ascending aorta 12, a second compliance element 335b and the descending aorta 14, and a third compliance element 335c lower in the descending aorta 14. Such compliance elements are illustrated as examples only; it should be understood that the implant device 300 may comprise any one or more of the illustrated compliance elements 335, which may be anchored in the left ventricle 3, within the aorta 16, or in any other anatomy.

Furthermore, compliance-enhancing implant devices of the present disclosure may include a plurality of tethered compliance elements separated by gap/extension connector(s) 339. That is, a first compliance element 335a, which may comprise a plurality of compliance balloons connected in a balloon chain as described in detail herein, may be coupled to a second group of compliance balloons 335b, wherein the first group 335a and the second group 335b are separated by a separation distance provided by the length of the connecting line/connector 339a. In some implementations, compliance-enhancing implant devices of the present disclosure include a first compliance element 335a that is disposed in the ascending aorta 12 and a second compliance element 335b that is disposed in the descending aorta 14, wherein a gap/separator connector 339a, which does not have compliance balloons associated therewith, separates and connects the compliance elements 335a, 335b. The gap/separator connector 339a can advantageously position the compliance element 335b outside of the area of the arterial branches 301 emanating from the aortic arch 13. For example, where ellipsoid compliance balloons are implemented in a compliance-enhancing implant device, it may be desirable to implant and/or configure such device such that the balloons thereof do not plug or otherwise obstruct the arterial branches that branch-off of the aorta or other branch/vessel relationship. For example, the blood vessels 301 include the brachiocephalic artery 27, the left common carotid artery 28, and the left subclavian artery 26. Obstruction of the ostia of such arteries can affect blood supply to the brain and/or other anatomy, potentially resulting in injury to the patient. Therefore, gap/separator connectors 339 may be implemented to herein as a means to avoid alignment of the compliance element 335, and/or balloons thereof, with branch blood vessel ostia (e.g., celiac, mesenteric, suprarenal, renal, lumbar, gonadal, or iliac arteries).

The compliance elements 335 comprise a set of compliance balloons, wherein such sets may consist of a plurality of balloons or a single balloon. That is, a ‘set’ of compliance elements, as described herein, may refer to a set of one or more balloons. A separate gap connector line/tether may connect between each adjacent set of compliance elements.

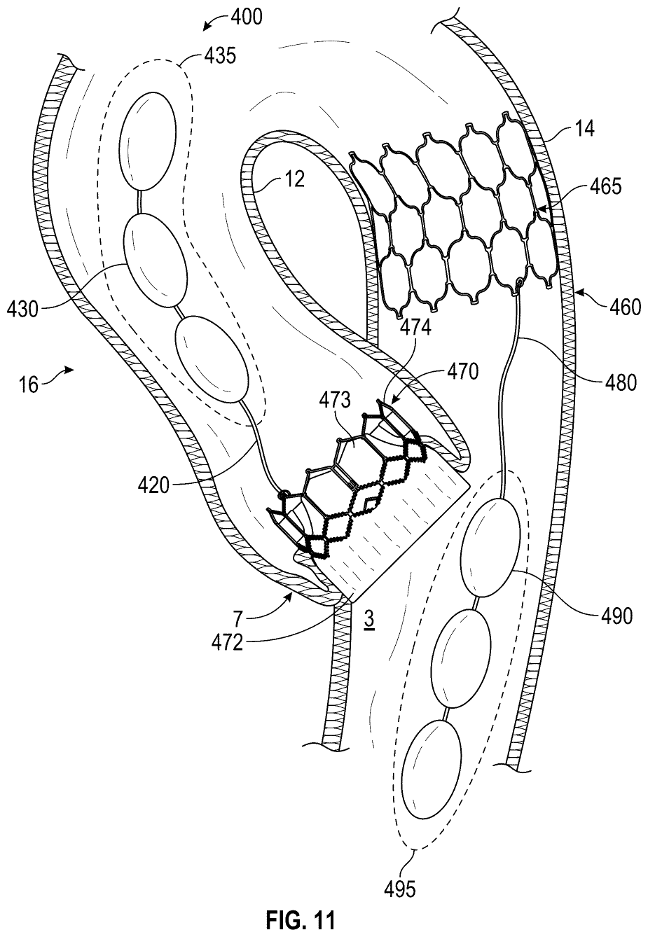

FIG. 11 shows a balloon-chain compliance-enhancing implant device 400 anchored/tethered to a prosthetic valve frame 471 in accordance with one or more examples. While some examples are presented herein wherein tethered compliance-enhancing balloon devices are tethered to tissue anchors configured to embed in tissue in the ventricle or target blood vessel, some examples provide for compliance-enhancing balloon devices (e.g., balloon-chain devices) that are tethered to other types of anchors, such as stents, frames, docking structures, or the like. The implant device is shown in FIG. 11 are examples of implant devices tethered to stents/frame-type implants.

In some examples, the compliance element 435 is tethered to a frame 471 of a prosthetic heart valve 470, which may be implanted within the annulus of a native aortic valve 7, or other valve. The prosthetic valve 470 may be any suitable or desirable prosthetic implant, such as a transcatheter-deliverable valve device having a frame configured to compress to a delivery profile for advancement through the patient's vasculature and expand at the implantation site (e.g., aortic valve annulus) to an expanded configuration in a secure position in the native valve anatomy. In some examples, the prosthetic valve 470 may be secured to the target anatomy using a docking device or structure. The prosthetic valve 470 may include additional features, such as a sealing skirt 472 and/or one-way leaflet 473 components, as shown.

The compliance element 435, which may comprise a balloon chain or singular balloon as described in detail herein, may be tethered to the frame 471 by a tether line 420. The tether line 420 may be attached to the frame 471 in any suitable or desirable manner, such as by tying, catching, clipping, crimping, or otherwise attaching the line 420 to the struts of the frame 471, which may comprise shape-memory metal or other at least partially rigid material. With the compliance element 435 tethered to the frame 471 of the replacement valve 470, the tether 420 may not need to pass through the aortic valve from the ventricle 3, but rather may be positioned entirely within the aorta 16 (or other target blood vessel).

While the frame 471 is illustrated as a component of a prosthetic heart valve 470, in other implementations, a compliance element 495 may be tethered to an anchor 465 which is not a component of a prosthetic valve (or other device). For example, the anchor 465 may comprise a stent or other frame/structure configured to be secured within the blood vessel, such as within the descending aorta 14 or other portion of the aorta or venous system. The compliance element 495, which may comprise a balloon chain of balloon elements 490, may be tethered by a tether line 480 to any portion, either internal and/or external, of the anchor frame 465. The frames 471, 465 may have any strut and/or cell design or configuration. In some implementations, a compliance element is tethered to a strut of a frame, as shown in the illustrated examples of FIG. 11. Although the anchor 465 is illustrated as a stent, other anchors may be implemented. In some implementations, the anchor 465 comprises a prosthetic valve device, which may include a stent frame and one or more leaflets or other valve mechanism (e.g., ball, stopper, etc.). Such valve may be a one-way valve that allows flow in the downstream direction of the blood vessel while preventing backflow.

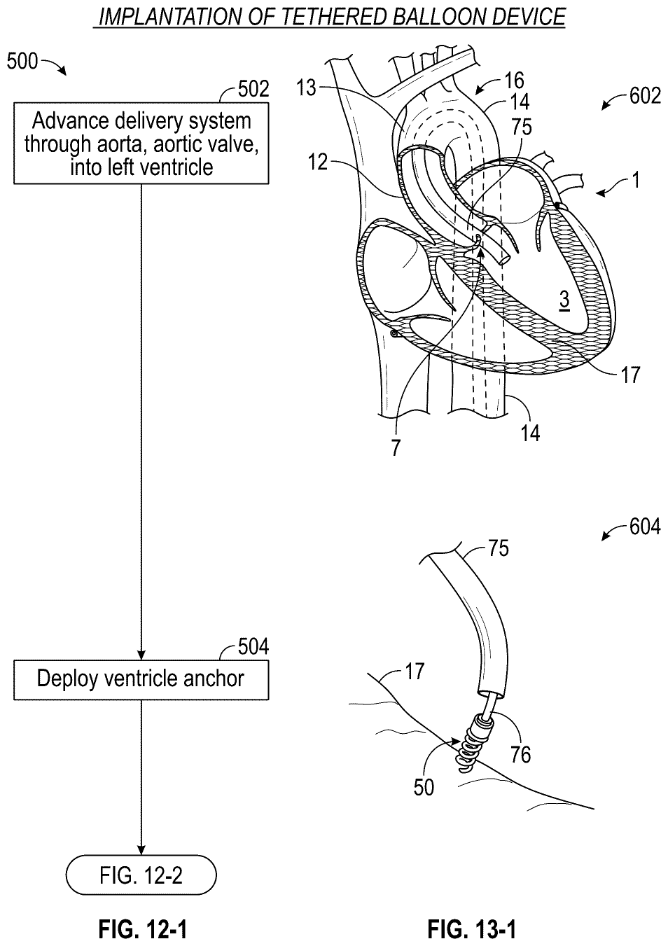

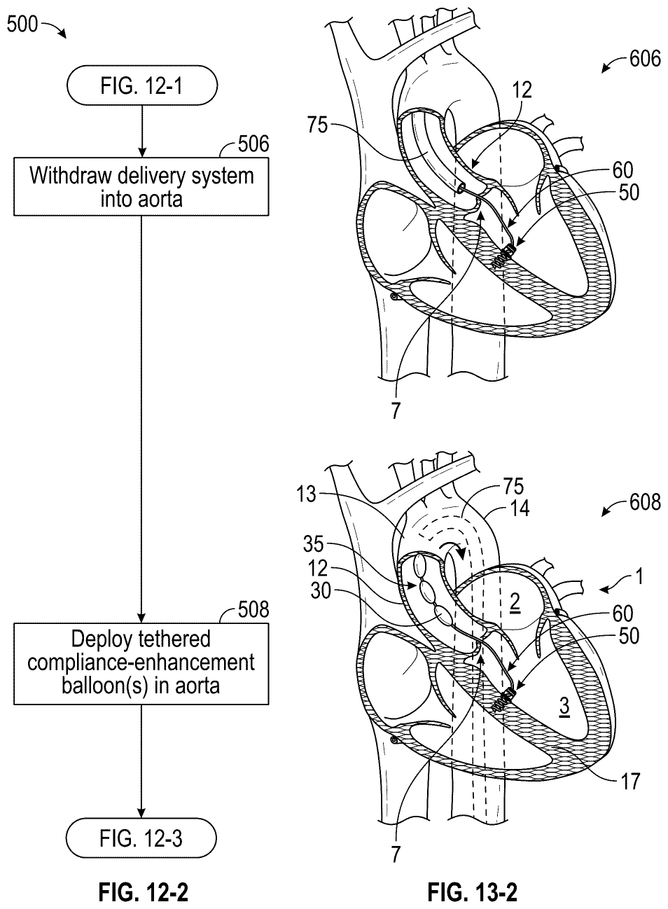

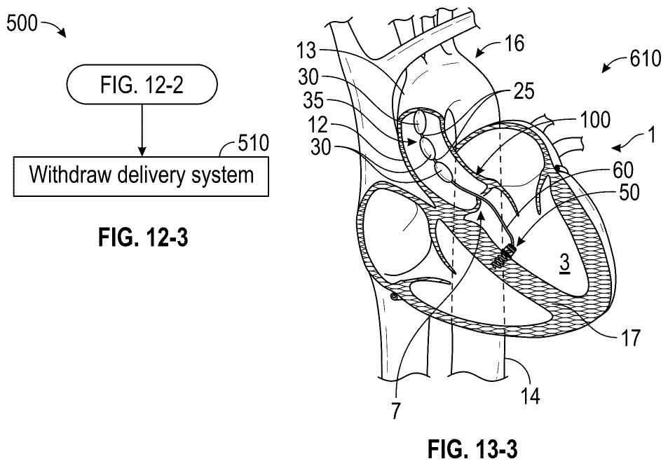

FIGS. 12-1, 12-2, and 12-3 illustrate a flow diagram for a process 500 for implanting a compliance-enhancing implant device 100 in accordance with one or more examples. FIGS. 13-1, 13-2, and 13-3 provide images of the compliance-enhancing implant device and certain anatomy corresponding to operations of the process 500 of FIGS. 12-1, 12-2, and 12-3 according to one or more examples.

At block 502, the process 500 involves advancing a delivery system 75 through the aorta 16 of a patient using a transcatheter procedure. For example, access to the aorta 16 may be through the femoral artery or other percutaneous vascular access. Block 502 further involves advancing the delivery system 75 through the aortic valve 7 and into the left ventricle 3. The delivery system 75 may be approximated to a target tissue wall, such as the ventricular septum 17.

At block 504, the process 500 involves deploying a tissue anchor 50 from the delivery system 75. The tissue anchor 50 may be embedded in the target tissue 17 in any suitable or desirable manner. For example, in some implementations, the tissue anchor 50 comprises a helical/corkscrew tissue-engagement portion configured to be embedded in the tissue 17. In some implementations, a driver tool 76 may be used to drive the tissue anchor 50 into the tissue 17, such as through rotation thereof. The delivery system 75 can include anchor-delivery shaft(s) (e.g., pusher(s)) for advancing the anchor(s) from a distal end of the delivery system 75; such shaft(s) can be used as a tissue anchor driver in some implementations.

At block 506, the process 500 involves withdrawing the delivery system 75 back into the aorta 12 (ascending aortic trunk) through the aortic valve 7. The tissue anchor 50 may have tethered thereto a tether line 60, which may likewise be deployed from the delivery system 75 as the delivery system 75 is withdrawn from the position/location of the tissue anchor 50.

At block 508, the process 500 involves deploying from the delivery system 75, in the aorta 12, a tethered compliance-enhancement element 35 comprising one or more compressible and/or re-shapable balloons 30 in accordance with details provided herein.

At block 510, the process 500 involves withdrawing the delivery system 75, thereby leaving behind the implant device 30, as shown in image 610. The implant device 30 remains in the cardiac and vascular anatomy with the tissue anchor 50 thereof embedded/anchored in/to the ventricular wall 17, with the tether line 60 passing from the ventricle 3, through the aortic valve 7, and into the aortic trunk 12. The compliance element 35 of the implant device 30 is disposed in the aorta 16 (e.g., at least partially within the ascending aortic trunk 12), the compliance element 35 comprising a plurality of compliance balloons 30 configured in a chain and connected by intermediate connector(s) 25, which may comprise any type of strut, line, cloth, polymer, or fabric segment coupled between adjacent balloons 30. As pressure increases in the aorta 16, such as in connection with the systolic phase of the cardiac cycle, such pressure exerts a compressive force on the balloons 30, thereby causing the balloons 30 to compress and/or reshape in a manner as to reduce a volume thereof. As the blood pressure decreases, such as in connection with diastolic phase of the cardiac cycle, the balloons 30 are permitted and/or inclined to revert to their expanded configuration shown in image 610, in which the balloons may have a generally-circular cross-sectional shape, at least with respect to a middle/medial portion thereof. The expanded state of the balloons 30 is associated with a greater volume thereof relative to the compressed/reshaped state assumed by the balloons during the high-pressure stage of the cardiac cycle. Such change in volume between the high- and low-pressure stages/phases can enhance compliance within the blood vessel 16, thereby reducing pulsatile flow and/or otherwise improving and/or evening-out the pressure waveform associated with the blood vessel.

While the process 500 is described as involving anchoring of the compliance implant device 100 in the ventricle 3, it should be understood that the process 500 may alternatively involve anchoring the compliance implant device 100 to tissue and/or to a structure deployed with in the aorta 16. For example, the process 500 may involve anchoring the tether 60 to a stent or prosthetic valve frame deployed within the aorta 16 and/or within the annulus of the native aortic valve 7, as described herein.

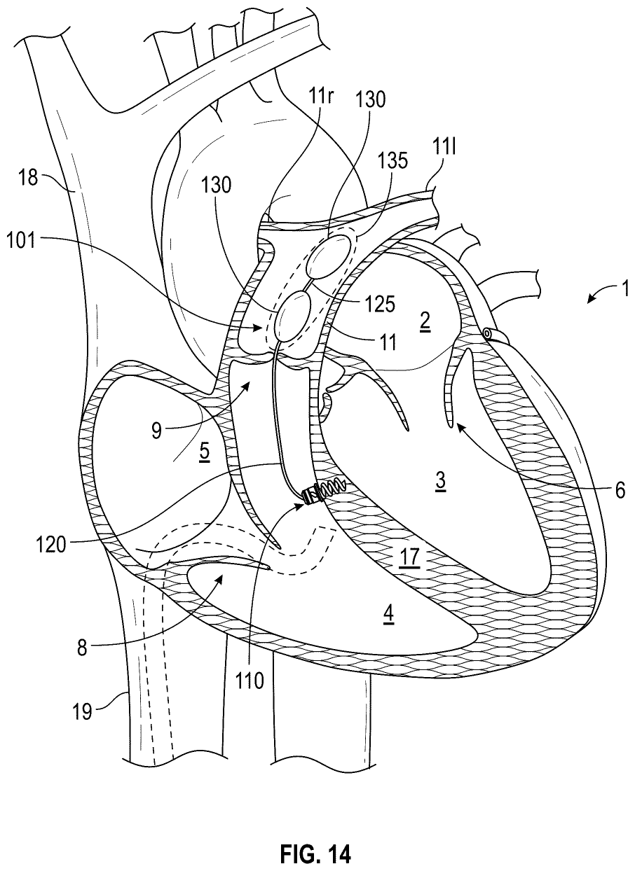

FIG. 14 shows a balloon-chain compliance-enhancing implant device 101 deployed in a pulmonary artery 11 and anchored in a right ventricle 4 in accordance with one or more examples. As referenced above, compliance-enhancing implant devices of the present disclosure can be deployed in any target vasculature in which increased compliance is desirable. FIG. 14 shows one such example, wherein an implant device 101 is implanted such that a compliance element 135 thereof is positioned within the pulmonary artery 11, such as within the pulmonary artery trunk, and/or at least partially within left 111 and/or right 11r branches of the pulmonary artery 11, which lead to left and right lungs, respectively. The compliance element 135 is tethered to an anchor 110, which may be configured to embed in tissue and/or expand within a blood vessel to secure the anchor therein (e.g., corkscrew anchor, stent frame). That is, although the tether 120 is shown as extending through the pulmonary valve 9 and into the right ventricle 4, in some implementations, the anchor 110 may take the form of a stent frame, prosthetic valve frame, or other device or structure positioned within the pulmonary artery 11 or other cardiac/vascular anatomy.

As referenced above, arterial hypertension represents a major cause of heart failure, wherein the afterload that the heart has to overcome to supply oxygen and nutrients to the body and organs is increased. Hypertension can lead to elevated pulse pressure and decreases cardiac output in some instances. In addition, pulmonary hypertension represents another heart failure with preserved ejection fraction (HFpEF) condition where increased pulmonary artery compliance can help alleviate the disease and potentially reduce hospital stays. With the compliance element 135 positioned within the pulmonary artery 11, the compliance of the pulmonary artery 11 can be increased, which can improve outcomes for a patient experiencing pulmonary hypertension.

The compliance element 135 may comprise any number of compliance balloons 130. That is, although the compliance element 135 is illustrated as a balloon chain including two balloons 130 connected by a connector 125, it should be understood that the compliance element 135 may include a single compliance balloon, which may or may not be elongated as described in detail above, or may include three or more balloons connected in a chain/series connection. The pressure and/or flow of blood in the right ventricle 4 and/or pulmonary artery 11 may serve to hold the compliance element 35 in place within the pulmonary artery 11.

The implant device 101 may be advanced to the pulmonary artery 11 through a transcatheter access. For example, a delivery sheath/catheter (shown in dashed-line) may be advanced through the venous system, such as through the inferior 19 or superior 18 vena cava, into the right atrium 5, through the tricuspid valve 8, into the right ventricle 4, and into the pulmonary artery 11 through the pulmonary valve 9. Alternatively, the device 101 may be implanted using an open-chest surgical procedure.

### Additional Description of Examples

Provided below is a list of examples, each of which may include aspects of any of the other examples disclosed herein. Furthermore, aspects of any example described above may be implemented in any of the numbered examples provided below.

Example 1: An implant device comprising a stent frame having an oval cross-sectional shape and a fluid-tight covering disposed on a portion of the stent frame.

Example 2: The implant device of any example herein, in particular example 1, wherein the oval cross-sectional shape includes first and second arched end walls on opposite major-axis ends of the stent frame, and first and second sidewalls on opposite minor-axis ends of the stent frame.

Example 1: An implant device comprising one or more balloons configured to decrease in volume in response to external fluid pressure above a threshold pressure level, and a tether line coupled to the one or more balloons.

Example 2: The implant device of any example herein, in particular example 1, further comprising a tissue anchor coupled to the tether line.

Example 3: The implant device of any example herein, in particular example 2, wherein the tissue anchor comprises a tissue-engagement portion configured to be embedded in biological tissue to secure the tissue anchor to the biological tissue.

Example 4: The implant device of any example herein, in particular example 3, wherein the tissue-engagement portion comprises a helical form, and the tissue anchor further comprises a drive head configured to be engaged and rotated by a driver instrument.

Example 5: The implant device of any of any example herein, in particular examples 2-4, wherein the tissue anchor comprises a cylindrical frame.

Example 6: The implant device of any example herein, in particular example 5, wherein the frame is a component of a prosthetic heart valve.

Example 7: The implant device of any example herein, in particular example 6, wherein the prosthetic heart valve is a transcatheter aortic valve replacement implant device.

Example 8: The implant device of any of any example herein, in particular examples 1-7, wherein the one or more balloons each are filled with compressible gas.

Example 9: The implant device of any of any example herein, in particular examples 1-8, wherein the one or more balloons each comprise a frame configured to hold open a vacuum-sealed chamber.

Example 10: The implant device of any of any example herein, in particular examples 1-9, wherein each of the one or more balloons is covered by a cover portion.

Example 11: The implant device of any example herein, in particular example 10, wherein the cover portion comprises air-tight polymer.

Example 12: The implant device of any example herein, in particular example 10 or claim 11, wherein the cover portion comprises air-tight fabric.

Example 13: The implant device of any of any example herein, in particular examples 10-12, wherein the one or more balloons comprises a first balloon and a second balloon connected by a portion of a covering that comprises the cover portion of each of the one or more balloons, the portion of the covering being fluid-sealed.

Example 14: The implant device of any of any example herein, in particular examples 1-13, wherein the one or more balloons consists of a single elongated balloon, the single elongated balloon having a shape of one of: an ellipsoid or a cylinder.

Example 15: The implant device of any of any example herein, in particular examples 1-14, wherein one or more balloons comprises a plurality of balloons including at least a first balloon and a second balloon.

Example 16: The implant device of any example herein, in particular example 15, wherein the plurality of balloons are connected in a chain arrangement.

Example 17: The implant device of any example herein, in particular example 16, wherein adjacent ones of the plurality of balloons are connected by connectors.

Example 18: The implant device of any example herein, in particular example 17, wherein the connectors comprise suture lines.

Example 19: The implant device of any example herein, in particular example 17 or claim 18, wherein the connectors comprise at least partially rigid struts.

Example 20: The implant device of any of any example herein, in particular examples 17-19, wherein the connectors comprise portions of a covering of the plurality of balloons.

Example 21: The implant device of any example herein, in particular example 20, wherein the portions of the covering are fluid-scaled, such that the portions of the covering can be split to disconnect adjacent ones of the plurality of balloons.

Example 22: The implant device of any of any example herein, in particular examples 1-21, wherein the tether line is sufficiently long to extend from a ventricular septum to an ascending aorta of a patient.

Example 23: The implant device of any example herein, in particular example 22, wherein the tether line is at least 3 cm in length.

Example 24: The implant device of any of any example herein, in particular examples 1-23, wherein the one or more balloons comprises at least three balloons connected in a chain.

Example 25: The implant device of any of any example herein, in particular examples 1-24, wherein the one or more balloons comprises a first set of balloons connected to a second set of balloons by a gap connector configured to span at least a portion of an aortic arch such that when the first set of balloons is positioned in an ascending aorta, the second set of balloons is positioned at least partially in a descending aorta.

Example 26: The implant device of any example herein, in particular example 25, wherein the gap connector is configured to prevent the second set of balloons from contacting ostia of main branches of the aortic arch.

Example 27: An implant device comprising a balloon chain including a plurality of compressible balloons connected in series, a tether coupled to a proximal-most one of the plurality of compressible balloons, and an anchor means coupled to a proximal end of the tether.

Example 28: The implant device of any example herein, in particular example 27, wherein the plurality of compressible balloons are filled with compressible gas.

Example 29: The implant device of any example herein, in particular example 27 or claim 28, wherein the plurality of compressible balloons are vacuum-filled.

Example 30: The implant device of any example herein, in particular example 29, wherein each of the plurality of compressible balloons comprises an ellipsoid frame covered with a fluid-tight cover portion.

Example 31: The implant device of any example herein, in particular example 30, wherein the ellipsoid frame comprises a structure designed to deflect radially inward when external pressure exceeds a threshold pressure level.

Example 32: The implant device of any example herein, in particular example 31, wherein the threshold pressure level is between 60-100 mmHg.

Example 33: The implant device of any of any example herein, in particular examples 27-32, wherein the anchor means comprises a corkscrew tissue anchor.

Example 34: The implant device of any of any example herein, in particular examples 27-33, wherein the anchor means comprises a stent frame.

Example 35: The implant device of any example herein, in particular example 34, wherein the stent frame is part of a prosthetic heart valve.

Example 36: The implant device of any of any example herein, in particular examples 27-35, wherein the plurality of compressible balloons comprises three or more balloons, and adjacent pairs of the three or more balloons are connected by longitudinal connectors.

Example 37: A method of controlling blood flow in a blood vessel, the method comprising providing a delivery system having disposed therein an implant device, the implant device comprising one or more compressible balloons, a tissue anchor, and a tether coupled between the tissue anchor and the one or more compressible balloons. The method further comprises advancing a distal portion of the delivery system through at least a portion of an aorta and through an aortic valve of the aorta into a left ventricle, deploying the tissue anchor from a distal portion of the delivery system, embedding the tissue anchor in tissue of the left ventricle, and deploying the one or more compressible balloons.

Example 38: The method of any example herein, in particular example 37, further comprising, after said embedding the tissue anchor in the tissue of the left ventricle, withdrawing the delivery system back through the aortic valve into the aorta, wherein said deploying the one or more compressible balloons involves deploying the one or more compressible balloons in the aorta.

Example 39: The method of any example herein, in particular example 38, wherein said deploying the one or more compressible balloons in the aorta involves deploying the one or more compressible balloons at least partially in an ascending portion of the aorta.

Example 40: The method of any example herein, in particular example 38 or claim 39, wherein said deploying the one or more compressible balloons in the aorta involves deploying the one or more compressible balloons at least partially in a descending portion of the aorta.

Example 41: The method of any of any example herein, in particular examples 38-40, wherein said deploying the one or more compressible balloons in the aorta involves deploying a first set of the one or more compressible balloons at least partially in an ascending portion of the aorta, and deploying a second set of the one or more compressible balloons at least partially in a descending portion of the aorta.

Example 42: The method of any of any example herein, in particular examples 37-41, wherein the one or more compressible balloons comprise ellipsoid forms filled with one of: compressible gas or vacuum.

Example 43: The method of any of any example herein, in particular examples 37-42, wherein the one or more compressible balloons comprises three or more balloons connected in a chain.

Example 44: A method of controlling blood flow in a blood vessel, the method comprising providing a delivery system having disposed therein an implant device, the implant device comprising an expandable frame, one or more compressible balloons, and a tether coupled between the frame and the one or more compressible balloons. The method further comprises advancing a distal portion of the delivery system within an aorta to a target portion of the aorta, deploying the frame in the target portion of the aorta, and proximally withdrawing the delivery system to deploy the tether and the one or more compressible balloons in the aorta.

Example 45: The method of any example herein, in particular example 44, wherein the expandable frame is a component of a prosthetic valve.

Example 46: The method of any example herein, in particular example 45, wherein said deploying the frame in the target portion of the aorta involves deploying the prosthetic valve in a native aortic valve annulus.

Example 47: The method of any of any example herein, in particular examples 44-46, wherein the frame is a stent.

Example 48: The method of any of any example herein, in particular examples 44-47, wherein the tether comprises a suture secured to one or more struts of the frame.

Example 49: The method of any of any example herein, in particular examples 44-48, wherein the one or more compressible balloons comprise ellipsoid forms filled with one of: compressible gas or vacuum.

Example 50: The method of any of any example herein, in particular examples 44-49, wherein the one or more compressible balloons comprises three or more balloons connected in a chain.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example. Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A compliance-enhancing medical implant device comprising:
a corkscrew tissue anchor including a tissue-engagement portion adapted to embed in an inner wall of a ventricle of a heart;
a tether line coupled to the corkscrew tissue anchor and dimensioned to pass through an aortic valve of the heart; and
one or more compressible, compliant balloons adapted to be disposed within an aorta associated with the heart and coupled to the corkscrew tissue anchor through the aortic valve via the tether line.

2. The compliance-enhancing medical implant device of claim 1, wherein:
the one or more compressible, compliant balloons comprises a plurality of ellipsoid balloons connected in series; and
a proximal-most one of the plurality of ellipsoid balloons is coupled to the tether line.

3. A compliance-enhancing medical implant device comprising:
one or more balloons that decrease in volume in response to external fluid pressure above a threshold pressure level, the one or more balloons being dimensioned for placement in a blood vessel;
a tether line coupled to at least one of the one or more balloons; and
a tissue anchor coupled to the tether line.

4. The compliance-enhancing medical implant device of claim 3, wherein the tissue anchor comprises a tissue-engagement portion configured to be embedded in biological tissue to secure the tissue anchor to the biological tissue.

5. The compliance-enhancing medical implant device of claim 4, wherein:
the tissue-engagement portion comprises a helical form; and
the tissue anchor further comprises a drive head configured to be engaged and rotated by a driver instrument.

6. The compliance-enhancing medical implant device of claim 3, wherein the tissue anchor comprises a cylindrical frame.

7. The compliance-enhancing medical implant device of claim 6, wherein the cylindrical frame is a component of a prosthetic heart valve.

8. The compliance-enhancing medical implant device of claim 3, wherein the one or more balloons each are filled with compressible gas.

9. The compliance-enhancing medical implant device of claim 3, wherein the one or more balloons each comprise a frame configured to hold open a vacuum-sealed chamber.

10. The compliance-enhancing medical implant device of claim 3, wherein the one or more balloons comprises a first balloon and a second balloon connected by a portion of a covering that covers each of the one or more balloons.

11. The compliance-enhancing medical implant device of claim 10, wherein the portion of the covering is fluid-sealed, such that the portion of the covering can be split to disconnect the first and second balloons.

12. The compliance-enhancing medical implant device of claim 3, wherein the one or more balloons consists of a single elongated balloon, the single elongated balloon having a shape of one of: an ellipsoid or a cylinder.

13. The compliance-enhancing medical implant device of claim 3, wherein the one or more balloons comprises a plurality of balloons including at least a first balloon and a second balloon connected in a chain arrangement by connecting lines.

14. The compliance-enhancing medical implant device of claim 3, wherein the one or more balloons comprises at least three balloons connected in a chain.

15. The compliance-enhancing medical implant device of claim 3, wherein the one or more balloons comprises a first set of balloons connected to a second set of balloons by a gap connector configured to span at least a portion of an aortic arch such that when the first set of balloons is positioned in an ascending aorta, the second set of balloons is positioned at least partially in a descending aorta.

16. A compliance-enhancing medical implant device comprising:
a balloon chain including a plurality of compressible balloons connected in series;
a tether coupled to a proximal-most one of the plurality of compressible balloons; and
an anchor means coupled to a proximal end of the tether.

17. The compliance-enhancing medical implant device of claim 16, wherein the plurality of compressible balloons are filled with compressible gas.

18. The compliance-enhancing medical implant device of claim 16, wherein the plurality of compressible balloons are vacuum-filled.

19. The compliance-enhancing medical implant device of claim 18, wherein each of the plurality of compressible balloons comprises an ellipsoid frame covered with a fluid-tight cover portion.

20. The compliance-enhancing medical implant device of claim 19, wherein the ellipsoid frame comprises a structure designed to deflect radially inward when external pressure exceeds a threshold pressure level.
